(19) United States
(12) Patent Application Publication
(10) Pub. No.: US 2024/0365889 A1
(43) Pub. Date: Nov. 7, 2024

(54) WEARABLE ORTHOPEDIC DEVICE FOR LOWER BODY POSTURE CORRECTION AND IMPROVED ERGONOMICS

(71) Applicant: IFGCure Holdings, LLC., Los Angeles, CA (US)

(72) Inventor: Stephen H. Liu, Los Angeles, CA (US)

(21) Appl. No.: 18/774,824

(22) Filed: Jul. 16, 2024

Related U.S. Application Data
(62) Division of application No. 16/827,614, filed on Mar. 23, 2020, now Pat. No. 12,048,338.
Application No. 16/827,614 is a continuation-in-part of application No. 29/714,472, filed on Nov. 22, 2019, now Pat. No. D921329.
Application No. 29/714,472 is a continuation-in-part of application No. 29/714,475, filed on Nov. 22, 2019, now Pat. No. D921330.
Application No. 29/714,475 is a continuation-in-part of application No. 16/368,588, filed on Mar. 28, 2019, now Pat. No. 10,736,364.
Application No. 16/368,588 is a continuation-in-part of application No. 16/147,639, filed on Sep. 29, 2018, now abandoned.
Application No. 16/147,639 is a continuation of application No. 16/125,453, filed on Sep. 7, 2018, now Pat. No. 10,721,975.
Application No. 16/125,453 is a continuation-in-part of application No. 16/057,558, filed on Aug. 7, 2018, now abandoned.
Application No. 16/057,558 is a continuation-in-part of application No. 16/029,567, filed on Jul. 7, 2018, now abandoned.
(60) Provisional application No. 62/649,542, filed on Mar. 28, 2018.

(51) Int. Cl. A41D 1/08

(52) U.S. Cl. CPC A41D 1/08; A41B 2300/22; A41B 2400/32

(57) ABSTRACT

The present disclosure is directed to a wearable orthopedic device for lower body posture correction and improve human ergonomics. Embodiments of the present disclosure include shorts that have specially positioned and tensioned panels for providing a wearer with a neutral pelvis. In certain embodiments, the device targets the body's proprioceptors to train the body to properly align itself.

### RELATED APPLICATION

This application is a divisional of U.S. patent application Ser. No. 16/827,614 entitled WEARABLE ORTHOPEDIC DEVICE FOR LOWER BODY POSTURE CORRECTION AND IMPROVED ERGONOMICS, filed on Mar. 23, 2020, which is a continuation-in-part of U.S. patent application Ser. No. 29/714,472 entitled ATHLETIC HIP SHORTS FOR MEN, filed on Nov. 22, 2019, a continuation-in-part of U.S. patent application Ser. No. 29/714,475 entitled ATHLETIC HIP SHORTS FOR MEN, filed on Nov. 22, 2019, and a continuation-in-part of U.S. patent application Ser. No. 16/368,588 entitled KINEMATIC BRA FOR POSTURE RECOVERY AND THORACIC MOBILITY filed Mar. 28, 2019, which is a continuation-in-part of U.S. patent application Ser. No. 16/147,639 entitled POSTURE RECOVERY THERAPEUTIC BRA filed Sep. 29, 2018, which is a continuation of U.S. patent application Ser. No. 16/125,453 entitled POSTURE, PERFORMANCE, RECOVERY (PPR) BRA filed Sep. 7, 2018, which is a continuation-in-part application of U.S. patent application Ser. No. 16/057,558 entitled POSTURE, PERFORMANCE, RECOVERY (PPR) BRA filed on Aug. 7, 2018, which is a continuation-in-part application of U.S. patent application Ser. No. 16/029,567 entitled POSTURE, PERFORMANCE, RECOVERY (PPR) BRA filed on Jul. 7, 2018, which claims priority to U.S. Provisional Patent Application No. 62/649,542, entitled POSTURAL RECOVERY BRA, filed Mar. 28, 2018. Each of the applications referred to in this paragraph are hereby incorporated by reference in their entirety as if set forth fully herein.

### FIELD OF THE DISCLOSURE

The present disclosure is related to the field of medical devices, especially wearable orthopedic devices for lower body posture correction and improved ergonomics.

### BACKGROUND OF THE DISCLOSURE

The importance of good posture and ergonomics is well known throughout the health care industry and work place ergonomics, and generally refers to the proper alignment of the body, specifically, to the relative static and dynamic positioning of the body, spine, pelvis, and its limbs. One important key factor to overall body posture is proper hip alignment, which is often associated with a neutral pelvis. A neutral pelvis is a position of the pelvis where the anterior superior iliac spine (ASIS) is in vertical alignment with the pubic crest. If the ASIS tilts forward in front of the pubic crest, then it creates an anterior pelvic tilt (APT), which can cause unwanted strain and curvature on the spine as well as chronic fatigue on the surrounding muscles and tendons. One tendon in particular that can be affected by APT is the iliotibial band (IT band) that runs along the outside of the upper leg from the ilium to the tibia (i.e., from the hip to the knee). Excess stretching of the IT band caused by APT can lead to inflammation of the IT band, known as IT band syndrome (ITBS).

A common known cause of APT is extended periods in a static seated position, such as sitting in a chair at work all day. Sitting for extended periods of time causes the body to adapt to a seated position. In a seated position, the hip flexor muscles (such as the tensor fasciae latae (TFL) and the adductor muscles) can shorten and the hip extensor muscles (such as the hamstrings and gluteus maximus) can lengthen, causing the pelvis to rotate forward toward the anterior of the body.

One way the body adapts to static and dynamic positioning is through proprioception. Two proprioceptors that play an important role in the flexibility of muscles are the muscle spindles and the golgi tendon organs (GTOs), which work together to regulate muscle stiffness and activation. Muscle spindles sense muscle length while GTOs sense muscle tension. When a muscle spindle senses that its agonist muscle is being stretched too far, it will send an afferent signal to the brain that the muscle needs to contract as a protective mechanism. The brain receives this afferent signal and sends an efferent signal back to the agonist muscle to activate and produce contraction. The muscle spindle can also send an afferent signal to the brain to inhibit an antagonist (i.e., opposing) muscle so that it will relax and not contribute to any further stretching of the agonist muscle. GTOs work opposite of muscle spindles to relax an agonist muscle when there is too much tension. A GTO can sense tension in an agonist muscle either when the muscle is contracted or being stretched. GTOs not only sense tension in the muscle but also the rate of tension. When a muscle is stretched slowly, the GTO will sense the slow rate of tension and temporarily inhibit the muscle spindle so that the muscle can relax, which is what occurs during a static stretch. When the body is in a static position that causes stretch on the muscles, the muscle spindles and GTOs begin to adapt to new resting positions such that they will only send signals to the brain under circumstances that deviate from the adapted muscle lengths and tensions.

When the body is in a seated position, the hip flexors shorten while the hip extensors lengthen. Although the hip extensors are being lengthened, the slow rate of stretching causes the GTO to inhibit the muscle spindle and allow the hip flexors to relax. During an extended period of static sitting, the muscle spindles begin to adjust their sensitivities such that their perception of the hip flexors becomes shorter than normal and their perception of the hip extensors becomes longer than normal. When the body stands back up, the bones and tendons (e.g., pelvis and IT band) attempt to return to a normal position, but this causes a stretch on what the muscle spindles perceive the adapted lengths of the hip flexors to be. Sensing a lengthening in the hip flexors, the muscle spindles send afferent signals to the brain to activate the hip flexors and inhibit the hip extensors, which causes the pelvis to tilt forward toward the anterior hip flexors and away from the posterior hip extensors.

A seated position can also shorten the adductor muscles and cause internal rotation of the femurs. When the femurs rotate internally, the femoral head pushes the pubic rami at the bottom of the pelvis backwards and the sacrum upwards, further tilting the pelvis anteriorly.

In addition to pelvic tilt, the shortening of the hip flexors and lengthening of the hip extensors also pulls the IT band in favor of its surrounding hip flexors such as the TFL and away from its surrounding hip extensors such as the hamstrings and gluteus maximus, which can cause unwanted tension and inflammation of the IT band. As such, APT has been associated with ITBS in addition to other postural related conditions.

Certain devices have attempted to correct APT by providing support to weak hip extensor and surrounding muscles to help the body achieve a neutral pelvis. One such device is disclosed in U.S. Pat. No. 7,670,205, explaining that “provision of a support on the piriform muscle leads to a large posture correction effect, and when it is used in combination with a support on the external oblique muscle, better effects can be expected.” However, providing support to weak muscles is merely a short fix for when the device is worn and does not offer an effective long-term solution that ultimately trains the body to stand in proper alignment even without the device. Other devices such as the one disclosed in U.S. Pat. No. 9,414,954 comprise numerous bulky and restricting straps that forcibly twist the body into alignment and require manual adjustment.

Dynamic positioning such as running, jumping, golfing, or athletic activity is also benefited by proprioceptive training. During dynamic movement, the GTOs may sense a much higher rate of tension than during static positioning, so they do not inhibit the muscle spindles in the same way described above. Muscle spindles may exhibit a dynamic response to stretch by increasing afferent firing rates to contract the stretched muscles, which can be counterproductive to the intended movement. For example, if the adductors are told to contract when they are being stretched by a dynamic movement, then this can cause internal rotation of the femurs, closing the hips.

Accordingly, there exists a need in the art for a simple device that can be comfortably worn as normal clothing or under normal clothing (for example, at work, during athletic activities, while training, during rehabilitation, for muscle recovery, or on a road trip) that trains the body to proprioceptively achieve better posture, a neutral pelvis, and less stress on the IT band. Accordingly, the present disclosure is aimed at solving these and other problems discussed below.

### SUMMARY OF THE DISCLOSURE

Certain embodiments of the present disclosure relate to wearable orthopedic devices for lower body posture correction. More specifically, certain embodiments are directed at men's and women's posture correction shorts. In some embodiments, the orthopedic devices have various carefully positioned panels with variable tensions to target certain areas of the body.

In some embodiments, the device targets areas of the body known to contribute to APT and ITBS by adjusting the sensitivities of proprioceptors such as muscle spindles and GTOS. Certain panels of the device may exert increased levels of tension along the IT band to help prevent it from being stretched by surrounding muscles. Other panels may exert increased levels of tension along the inseam to help prevent internal rotation of the femurs.

It is an object of embodiments of the present disclosure to reduce excessive strain on a wearer's IT band.

It is another object of embodiments of the present disclosure to reduce internal rotation of a wearer's femurs.

It is yet another object of embodiments of the present disclosure to correct a wearer's posture by training the wearer's muscles and proprioceptors.

These and other further features and advantages provided in this disclosure would be apparent to those skilled in the art from the following detailed description, taken together with the accompanying drawings.

### BRIEF DESCRIPTION OF THE DRAWINGS

FIG. 1 is a front view of the exterior of an orthopedic device according to another embodiment of the disclosure.

FIG. 2 is a back view of the exterior of an orthopedic device according to another embodiment of the disclosure.

FIG. 3 is a right view of the exterior of an orthopedic device according to another embodiment of the disclosure.

FIG. 4 is a left view of the exterior of an orthopedic device according to another embodiment of the disclosure.

FIG. 5 is a bottom view of the exterior of an orthopedic device according to another embodiment of the disclosure.

FIG. 6 is a front view of the interior of an orthopedic device according to another embodiment of the disclosure.

FIG. 7 is a back view of the interior of an orthopedic device according to another embodiment of the disclosure.

FIG. 8 is a right view of the interior of an orthopedic device according to another embodiment of the disclosure.

FIG. 9 is a left view of the interior of an orthopedic device according to another embodiment of the disclosure.

FIG. 10 is a bottom view of the interior of an orthopedic device according to another embodiment of the disclosure.

### DETAILED DESCRIPTION OF THE DISCLOSURE

Throughout this disclosure, the embodiments illustrated should be considered as exemplars, rather than as limitations on the present disclosure. As used herein, the term “invention,” “device,” “apparatus,” “method,” “disclosure,” “present invention,” “present device,” “present apparatus,” “present method,” or “present disclosure” refers to any one of the embodiments of the disclosure described herein, and any equivalents. Furthermore, reference to various features of the “invention,” “device,” “apparatus,” “method,” “disclosure,” “present invention,” “present device,” “present apparatus,” “present method,” or “present disclosure” throughout this document does not mean that all claimed embodiments or methods must include the reference features.

It is also understood that when an element or feature is referred to as being “on” or “adjacent” to another element or feature, it can be directly on or adjacent the other element or feature or intervening elements or features may also be present. In contrast, when an element is referred to as being “directly on” or extending “directly onto” another element, there are no intervening elements present. Additionally, it is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

Furthermore, relative terms such as “inner,” “outer,” “upper,” “top,” “above,” “lower,” “bottom,” “beneath,” “below,” and similar terms, may be used herein to describe a relationship of one element to another. Terms such as “higher,” “lower,” “wider,” “narrower,” and similar terms, may be used herein to describe angular relationships. It is understood that these terms are intended to encompass different orientations of the elements or system in addition to the orientation depicted in the figures.

Although the terms first, second, third, etc., may be used herein to describe various elements, components, regions, and/or sections, these elements, components, regions, and/or sections should not be limited by these terms. These terms are only used to distinguish one element, component, region, or section from another. Thus, unless expressly stated otherwise, a first element, component, region, or section discussed below could be termed a second element, component, region, or section without departing from the teachings of the present disclosure. As used herein, the term “and/or” includes any and all combinations of one or more of the associated list items.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. For example, when the present specification refers to “an” assembly, it is understood that this language encompasses a single assembly or a plurality or array of assemblies. It is further understood that the terms “comprises,” “comprising,” “includes,” and/or “including” when used herein, specify the presence of stated features, integers, steps, operations, elements, and/or components, but do not preclude the presence or addition of one or more other features, integers, steps, operations, elements, components, and/or groups thereof.

Embodiments as described in the present disclosure can be described herein with reference to view illustrations, some of which are schematic in nature. As such, the actual thickness of elements can be different, and variations from the shapes of the some of the illustrations as a result, for example, of manufacturing techniques and/or tolerances are expected. Thus, the elements illustrated in the some of the figures are schematic in nature and their shapes are not intended to illustrate the precise shape of a region and are not intended to limit the scope of the disclosure.

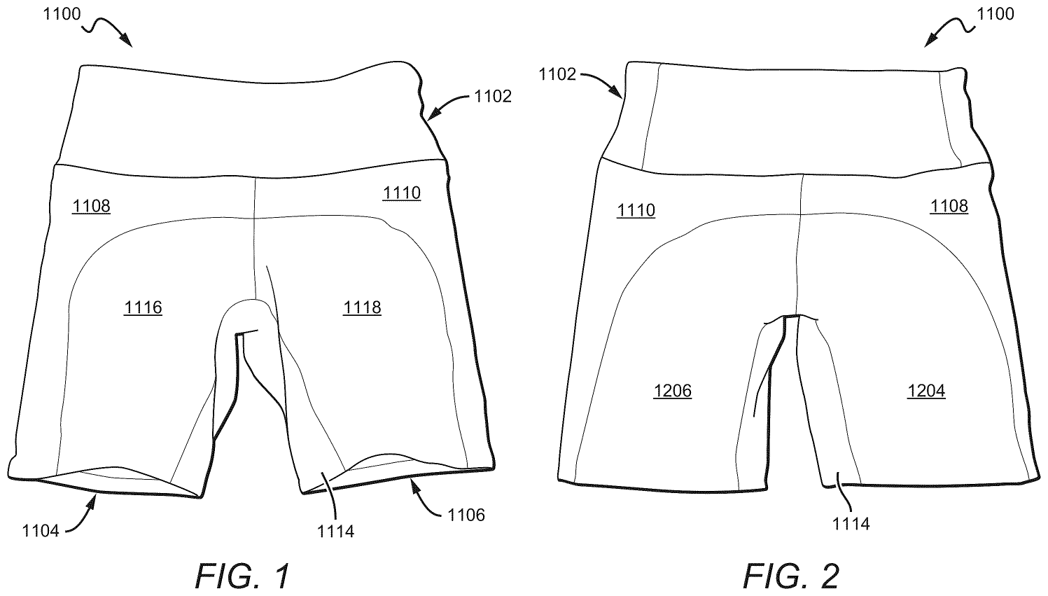

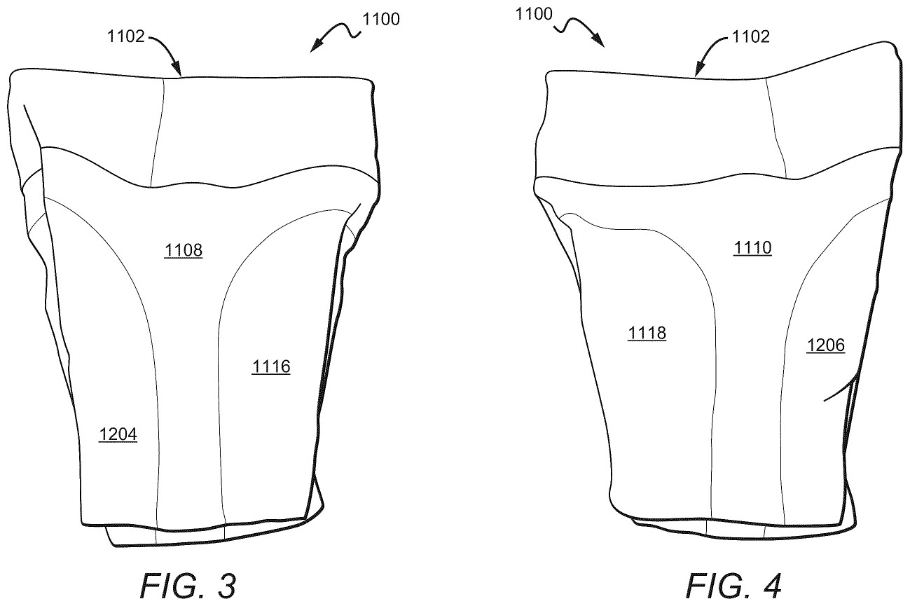

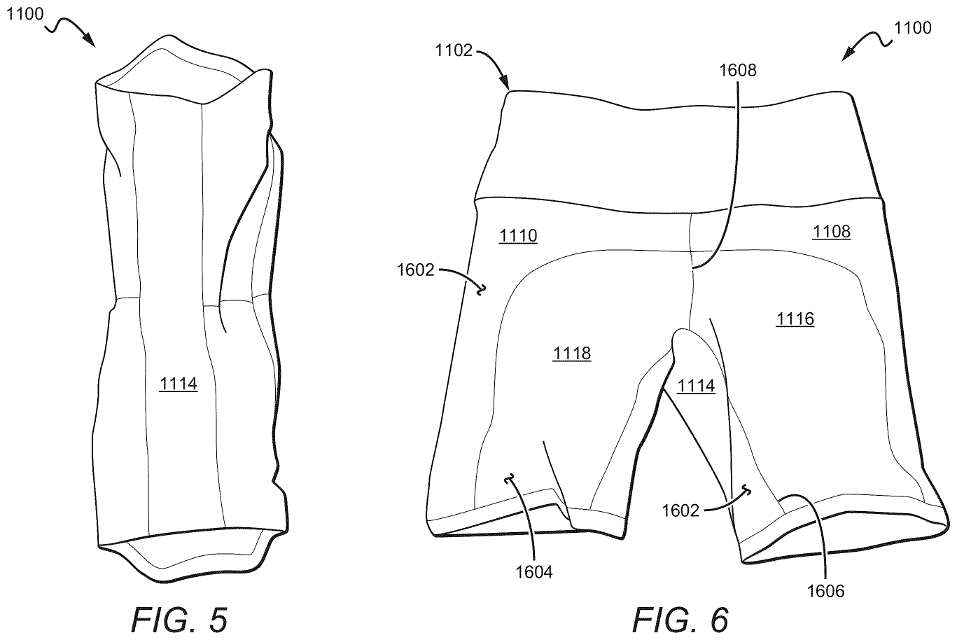

FIGS. 1-5 show various views of the exterior of an orthopedic device 1100 according to another embodiment of the disclosure. As shown, device 1100 is a pair of women's posture correction shorts. In the embodiment shown, device 1100 comprises a waistband 1102, right and left legs 1104, 1106, and a series of panels. As shown in FIGS. 1-5, the panels may include: a right side panel 1108 that runs vertically from the bottom of the right side of right leg 1104 to the right side of waistband 1102 and curves to extend horizontally along waistband 1102 around the anterior and posterior of device 1100; a left side panel 1110 that runs vertically from the bottom of the left side of left leg 1106 to the left side of waistband 1102 curves to extend horizontally along waistband 1102 around the anterior and posterior of device 1100 to connect with right side panel 1108 at the center of the anterior and posterior of device 1100 below waistband 1102; an inseam panel 1114 running along the bottom of device 1100; right and left front panels 1116, 1118 on the anterior of the device; and right and left back panels 1204, 1206 on the posterior of the device.

In the embodiment shown in FIGS. 1-5, inseam panel 1114 runs along the bottom of device 1100 from the bottom of the left side of right leg 1104 to the bottom of the right side of left leg 1106 and directly connects between right and left front panels 1116, 1118 and right and left back panels 1204, 1206. Right and left front panels 1116, 1118 are directly connected to the anterior of right and left side panels 1108, 1110, respectively, and extend to the bottoms of the front sides of right and left legs 1104, 1106, respectively. Right and left back panels 1204, 1206 are directly connected to the posterior of right and left side panels 1108, 1110, respectively, and extend to the bottoms of the back sides of right and left legs 1104, 1106, respectively.

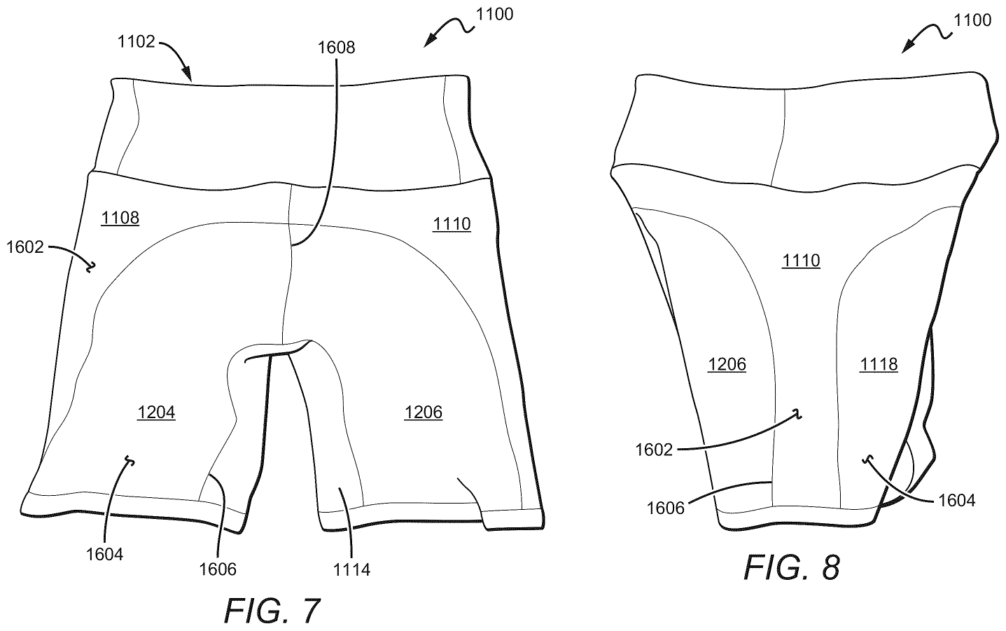

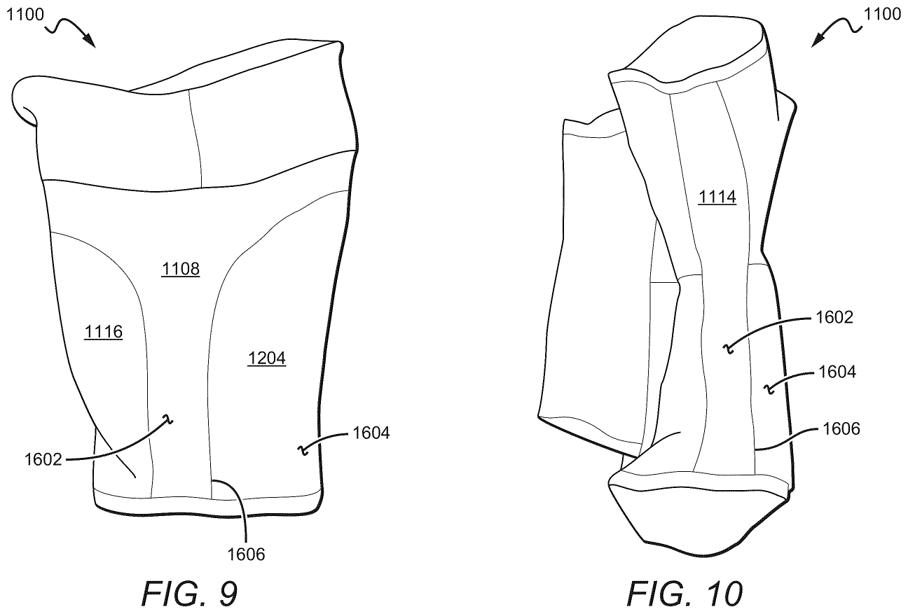

FIGS. 6-10 show various views of the interior of device 100 according to one embodiment of the disclosure. As shown in FIGS. 6-10, right side panel 1108, left side panel 1110, and inseam panel 1114 may comprise a double-layer fabric 1602 with an interior fabric and an exterior fabric, while right front panel 1116, left front panel 1118, right back panel 1204, and left back panel 1206 may comprise a single-layer fabric 1604. In certain preferred embodiments, the interior fabric of double-layer fabric 1602 comprises a mesh material. The double-layer fabric 1602 of the right side panel 1108 and left side panel 1110 is specifically targeted at providing increased tension along the IT bands. Providing tension along the IT bands helps the surrounding muscles resist lengthening and shortening and remain at a normal length, which prevents the muscle spindles and GTOs from adapting to abnormal positions. Thus, when the body begins to stand, it does not feel a proprioceptive need to contract the hip flexors and relax the hip extensors because it will perceive these muscles to be at a normal length. Without hip flexor contraction, the IT band is also not stretched and is able to maintain a normal position. Likewise, the inseam panel 1114 provides tension near the adductors so that they will resist shortening and internally rotating the femurs. This, too, will help prevent anterior tilting of the pelvis by keeping the public rami in proper alignment. These embodiments allow for right front panel 1116, left front panel 1118, right back panel 1204, and left back panel 1206 to have a greater elasticity than right side panel 1108, left side panel 1110, and inseam panel 1114. Accordingly, each connection along an edge of right side panel 1108, left side panel 1110, and inseam panel 1114 may comprise a first stitch 1606 that is heavier than a second stitch 1608 provided along each connection not along one of these edges in order to better attach the panels that comprise additional and less elastic fabrics. In some embodiments, first stitch 1606 is a flat lock stitch.

In addition to working prophylactically to prevent APT, device 1100 can also work therapeutically to retrain the body once it is already in APT. If the hip flexors have already been shortened from long periods of sitting, right side panel 1108, left side panel 1110, and inseam panel 1114 will provide tension to pull the hip flexors such as the TFL and adductors back into a normal length. This will cause the body's proprioception to work the same as described above, but now in favor of a neutral pelvis. When the body is already out of alignment, device 1100 will create a static stretch on the hip flexors, causing the GTOs to inhibit the muscle spindles and relax the hip flexors. This will, in turn, cause the muscle spindles to readjust to a lengthened position of the hip flexors so that when the body is in a standing position, the muscle spindles will not perceive a need to contract these muscles and pull the pelvis forward. In this way, the body's proprioception is being retrained to perceive a neutral pelvis position as normal again. Retraining the body's proprioception provides lasting effects that can remain even when device 1100 is not worn.

Device 1100 can also help with dynamic positioning such as running or athletic activity. By maintaining the normal lengths of certain muscles like the adductors, the muscle spindles will not feel a lengthening and tell the body to contract. This can be beneficial, for example, by allowing the hips to open up without internal rotation by the femurs.

It is understood that embodiments presented herein are meant to be exemplary. Embodiments of the present disclosure can comprise any combination or compatible features shown in the various figures, and these embodiments should not be limited to those expressly illustrated and discussed.

Although the present disclosure has been described in detail with reference to certain configurations thereof, other versions are possible. Further, none of the elements or features discussed herein should be construed as necessary, critical, or essential for any particular embodiment of the present disclosure. Therefore, the spirit and scope of the disclosure should not be limited to the versions described above. The foregoing is intended to cover all modifications and alternative constructions falling within the spirit and scope of the disclosure as expressed in the appended claims, wherein no portion of the disclosure is intended, expressly or implicitly, to be dedicated to the public domain if not set forth in the claims.

What is claimed is:

1. A wearable orthopedic device, comprising:
a waistband;
a right leg and a left leg;
a right side panel running vertically from a bottom of a right side of said right leg to a right side of said waistband and curving to extend horizontally along said waistband around the anterior and posterior of said device;
a left side panel running vertically from a bottom of a right side of said left leg to a left side of said waistband and curving to extend horizontally along said waistband around the anterior and posterior of said device, wherein said left side panel and said right side panel are directly connected at a central location on the anterior of said device directly below said waistband and a central location on the posterior of said device directly below said waistband;
a right front panel on the anterior of said device directly connected along said right side panel and extending to a bottom of a front side of said right leg;
a left front panel on the anterior of said device directly connected along said left side panel and extending to a bottom of a front side of said left leg, wherein said left front panel and said right front panel are directly connected at a central location on the anterior of said device directly below said anterior connection of said right and left side panels;
a right back panel on the posterior of said device directly connected along said right side panel and extending to a bottom of a back side of said right leg;
a left back panel on the posterior of said device directly connected along said left side panel and extending to a bottom of a back side of said left leg, wherein said left back panel and said right back panel are directly connected at a central location on the posterior of said device directly below said posterior connection of said right and left side panels;
an inseam panel running along the bottom of said device from a bottom of a left side of said right leg to a bottom of a right side of said left leg, said inseam panel directly connected between said right and left front panels and said right and left back panels,
wherein said right side, left side, and inseam panels comprise a double-layer fabric with an interior fabric and an exterior fabric, said interior fabric comprising a mesh material,
wherein said right front, left front, right back, and left back panels comprise a single-layer fabric such that said right front, left front, right back, and left back panels have a greater elasticity than said right side, left side, and inseam panels,
wherein each connection along an edge of said right side panel, left side panel, or inseam panel comprises a first stitch and each connection not along an edge of said right side panel, left side panel, or inseam panel comprises a second stitch, wherein said first stitch is heavier than said second stitch.

2. The device of claim 1, wherein said double-layer fabric on said right side panel and said left side panel is configured to reduce stretch on a wearer's IT band.

3. The device of claim 1, wherein said double-layer fabric on said inseam panel is configured to reduce internal rotation of a wearer's femurs.

4. The device of claim 1, wherein said first stitch is a flat-lock stitch.

5. A wearable orthopedic device, comprising:
a waistband;
a right leg comprising a ride side panel, a right front panel, and a right back panel;
a left leg comprising a left side panel, a left front panel, and a left back panel;
an inseam panel;
wherein said right front, left front, right back, and left back panels comprise a single-layer fabric, and said right side, left side, and inseam panels comprise a double-layer fabric with an interior fabric and an exterior fabric, wherein said right side panel runs vertically from a bottom of a right side of said right leg to a right side of said waistband and curves to extend horizontally along said waistband around the anterior and posterior of said device, and wherein said left side panel runs vertically from a bottom of a left side of said left leg to a ride side of said waistband and curves to extend horizontally along said waistband around the anterior and posterior of said device, and wherein said single-layer fabric has a greater elasticity than said double-layer fabric, and
wherein seams along edges of said right side panel, said left side panel, and said inseam panel to other portions of said device comprise a first stitch type, and seams not along an edge of said right side panel, said left side panel, and said inseam panel comprise a second stitch type, wherein said first stitch type comprises a higher thread density than said second stitch type.

6. The device of claim 5, wherein said left side panel and said right side panel are directly connected at a central location on the anterior of said device directly below said waistband and a central location on the posterior of said device directly below said waistband.
